(19) United States
(12) Patent Application Publication
(10) Pub. No.: US 2016/0128721 A1
(43) Pub. Date: May 12, 2016

(54) SURGICAL INSTRUMENT

(71) Applicant: Jan PERSSON, Lund (SE)

(72) Inventor: Jan PERSSON, Lund (SE)

(21) Appl. No.: 14/897,382

(22) PCT Filed: Jul. 9, 2014
(86) PCT No.: PCT/EP2014/061932

§ 371 Date: Dec. 10, 2015

(30) Foreign Application Priority Data
Jul. 5, 2013 (SE) 1350843-7

(51) Int. Cl. A61B 17/34; A61B 17/02

(52) U.S. Cl. CPC A61B 17/3423; A61B 17/0218; A61B 2217/005; A61B 2217/007

(57) ABSTRACT

A surgical instrument is disclosed having a proximal end and a distal end. The surgical instrument includes a hook portion arranged at said distal end for retracting tissue and/or organs during surgery. The surgical instrument further includes an elongate tube adapted for suction and/or irrigation, said tube extending essentially along a length of said surgical instrument.

### TECHNICAL FIELD OF THE INVENTION

The present invention relates to a surgical instrument having a proximal end and a distal end, comprising a hook portion arranged at said distal end for retracting tissue and/or organs during surgery.

### BACKGROUND ART

During surgery, it is generally necessary to be able to retract tissue or organs to provide access to areas of interest. To this end, hooks and retractors of various types are used. An example of a retractor is the so called Kelly retractor.

When performing keyhole surgery, such as laparoscopy, the surgical instruments used are introduced in the body of the patient via trocars inserted through the skin. In order to be able to retract tissue and organs, hooks and retractors of a small diameter are used. Examples of laparoscopic retractors may be found, e.g., in WO 2008/068405. The diameter of the hook or retractor is adapted to the inner diameter of the trocar. If suction or irrigation is needed, the retractor has to be removed, out through the trocar, and a suction/irrigation tube inserted instead. When suction or irrigation has been finished, the suction tube is removed through the trocar, and the retractor is once more inserted. This takes time, and the tissue or organ that was earlier retracted has to be located again.

### SUMMARY OF THE INVENTION

It is an object of the invention to provide a surgical instrument that simplifies surgery as compared to prior art instruments.

According to the invention, these and other objects are achieved, in full or at least in part, by a surgical instrument having a proximal end and a distal end, comprising a hook portion arranged at said distal end for retracting tissue and/or organs during surgery, and further comprising an elongate tube adapted for suction and/or irrigation, said tube extending essentially along a length of said surgical instrument. With such an instrument, it is possible to retract tissue or organs also during suction and/or irrigation, without having to add another instrument. In the following, where reference is made to suction, irrigation is equally applicable. Such an instrument is particularly useful in connection with keyhole surgery, such as laparoscopy or robot assisted laparoscopy, where the site of the surgery is accessed via trocars inserted through the skin of the patient. The number of trocars has to be limited, for the assistant surgeon ideally to just one. With current retractors, the retractor has to be removed from the surgical site, out trough the trocar, to allow access with a suction tube when suction is needed. When suction has been performed, the suction tube is removed, and the retractor once more inserted through the trocar. However, the tissue or organ that was earlier retracted will then have returned to its normal position, and the operator of the retractor has to locate the correct spot again to resume retraction of the tissue or organ. Moreover, a simultaneous retraction and suction is often crucial for a good result and safe surgery. With the surgical instrument of the invention, it is possible to perform suction, while a tissue or organ is still being retracted, thereby saving time and reducing the risk of injury from moving the retractor around in search of the correct location, as well as enabling simultaneous suction and irrigation in case of an ongoing bleeding, which is a potentially dangerous situation. The elongated tube shape of the surgical instrument of the invention makes it useful for keyhole surgery, such as laparoscopy. The surgical instrument of the invention is particularly useful for robotic surgery, where a single assistant trocar is usually advocated.

In a preferred embodiment, the surgical instrument is essentially straight. An essentially straight shape is necessary for allowing insertion of the instrument through a trocar. An essentially straight shape further facilitates the use of the instrument since there are no protruding parts, deviating considerably from the elongated tube shape, at the proximal end of the instrument which may be in the way during surgery.

In one embodiment, the tube extends from the proximal end to the distal end of the surgical instrument, enabling suction in front of the retracted tissue. If the distal end of the surgical end is formed with an atraumatic blunt shape, simultaneous compression of a bleeding vessel with the proximal end is also enabled.

The surgical instrument may further comprise a coupling portion arranged at the proximal end, for connection to a suction and/or irrigation device, said tube forming a suction and/or irrigation tube. In this manner, the surgical instrument may be connected to a suction and/or irrigation device and used instead of a suction and/or irrigation tube that would otherwise be used with that suction and/or irrigation device.

The coupling portion may comprise a rotary coupling for allowing rotation of the surgical instrument in relation to the suction and/or irrigation device. Such a rotary coupling makes it possible to move the surgical instrument around freely, regardless of the orientation of a handle of the suction and/or irrigation device.

In an embodiment, the hook portion is arranged adjacent the distal end of the surgical instrument and the tube extends distally beyond said hook portion. With such an arrangement of the hook portion and the distal tube end, suction and/or irrigation may easily be performed while a tissue or organ is retracted. The length of the tube that extends distally beyond the hook portion may be varied, such that, e.g., the entire hook portion is proximal of the distal end of the surgical instrument.

In another embodiment, the tube extends from the proximal end of the surgical instrument to a location adjacent the distal end. Thereby suction and/or irrigation may be enabled at the end of the surgical instrument, but also at positions immediately proximal or distal of the end, with simultaneous retraction.

The hook portion may extend from an outer end of the tube to the distal end of the surgical instrument. With such an arrangement of the hook portion, it is possible to make the hook portion larger, without making the total diameter of the surgical instrument unnecessarily large, such that the surgical instrument may fit in trocars of different diameters.

The tube may be adapted for insertion of a suction and/or irrigation tube. In this manner, the suction and/or irrigation tube of a conventional suction and/or irrigation device may be inserted in the tube and used for suction and/or irrigation, while the hook portion of the surgical instrument retracts a tissue or organ.

The hook portion may comprise an opening allowing passage of a suction and/or irrigation end of the suction and/or irrigation tube. Thereby, the suction and/or irrigation tube may gain easy access to a location where suction and/or irrigation is needed, while the hook portion securely retracts a tissue or organ.

The surgical instrument may further comprise a retaining device arranged to fix a longitudinal position of said suction and/or irrigation tube inside said tube. This makes it possible to keep the suction and/or irrigation tube inserted in the tube of the surgical instrument also when suction and/or irrigation is not needed. Once suction and/or irrigation is completed, the suction and/or irrigation tube may simply be retreated a short distance into the tube of the surgical instrument and held in position there until suction and/or irrigation is once more needed.

The surgical instrument may further comprise a sealing device for sealing said tube around said suction and/or irrigation tube. Thereby, the distal end of the surgical instrument may be isolated from the surrounding atmosphere. In this manner, the distal end of the surgical instrument may be inserted without loss of the pneumoperitoneum, i.e. the increased gas pressure inside the abdomen that is applied in order to create space for surgery.

The sealing device may be arranged to seal the tube of the surgical instrument when the suction and/or irrigation tube is removed from the tube of the surgical instrument. In this manner, sealing of the tube of the surgical instrument may be ensured when the suction and/or irrigation tube is inserted, as well as when the suction and/or irrigation tube is removed.

The sealing device may further be arranged to fix a longitudinal position of said suction and/or irrigation tube inside said tube. With this arrangement, sealing and fixing may be achieved by one and the same device.

The hook portion may have a saddle shape. Such a shape is useful for ensuring secure retraction, for safe application of pressure on bleeds, and may present a safe, rounded contour reducing the risk of injury when the surgical instrument is inserted or moved around.

The surgical instrument may comprise a measuring scale formed on said tube. This is particularly useful during keyhole surgery, where the surgical site is viewed via camera. The image of the surgical site is generally enlarged, in order to provide more detail to the surgeon. Depending on the location of the camera, the enlargement varies. However, with a varied enlargement, it is often difficult to retain a proper sense of distance. A measuring scale on the surgical instrument, which is visible in the image of the surgical site, may assist the surgeon in assessing distances. The measuring scale may, for instance, be coated onto the surgical instrument or engraved in the surface of the surgical instrument.

The surgical instrument may be made of a medical grade metal material. Suitable metals are available that can easily be formed to the desired shape and that have good structural strength and wear resistance properties.

The surgical instrument may be made of or be coated with an electrically non-conductive, medical grade material. This makes it possible to safely cauterize bleeding vessels electrically, or use electrodissection, even close to the surgical instrument.

Other objectives, features and advantages of the present invention will appear from the following detailed disclosure, from the attached claims, as well as from the drawings. It is noted that the invention relates to all possible combinations of features.

Generally, all terms used in the claims are to be interpreted according to their ordinary meaning in the technical field, unless explicitly defined otherwise herein. All references to “a/an/the [element, device, component, means, step, etc.]” are to be interpreted openly as referring to at least one instance of said element, device, component, means, step, etc., unless explicitly stated otherwise. The steps of any method disclosed herein do not have to be performed in the exact order disclosed, unless explicitly stated.

As used herein, the term “comprising” and variations of that term are not intended to exclude other additives, components, integers or steps.

### BRIEF DESCRIPTION OF THE DRAWINGS

The invention will be described in more detail with reference to the appended schematic drawings, which show examples of currently preferred embodiments of the invention.

FIG. 1 is a side view of a surgical instrument according to an embodiment.

FIG. 2 is a side view of a surgical instrument according to another embodiment.

FIG. 3a is a detail view of a distal end of the surgical instrument of FIG. 1.

FIG. 3b is a detail view of a proximal end of the surgical instrument of FIG. 1.

FIG. 4a is a detail view of a distal end of the surgical instrument of FIG. 2.

FIG. 4b is a detail view of a proximal end of the surgical instrument of FIG. 2.

### DETAILED DESCRIPTION OF PREFERRED EMBODIMENTS OF THE INVENTION

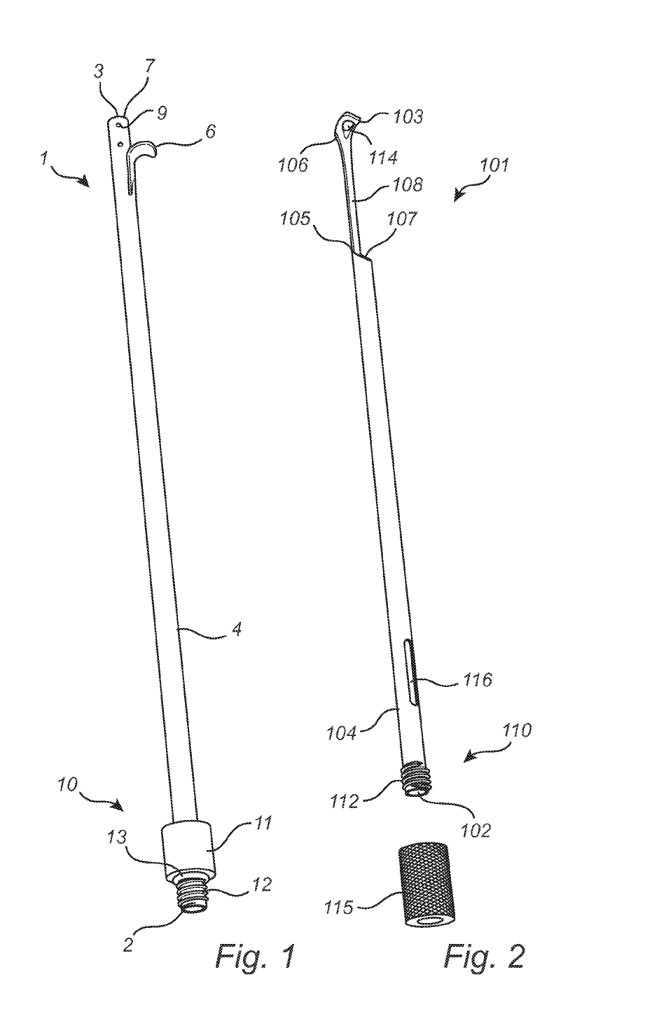

In FIG. 1, a surgical instrument 1 according to a first embodiment is shown. The surgical instrument 1 has a proximal end 2 and a distal end 3. The proximal end 2 is intended to be held by an operator during surgery, whereas the distal end 3 is intended to be inserted in a trocar or other surgical incision. The surgical instrument 1 is generally made up of a tube 4, which extends from the proximal end 2 of the surgical instrument 1 to the distal end 3. An outer end 7 of the tube 4 thus coincides with the distal end 3 of the surgical instrument 1. In the embodiment shown, the outer end 7 of the tube 4 is perpendicular to a longitudinal direction of the tube 4. As mentioned above, the proximal end 2 of the instrument 1 is intended to be held by an operator. Hence, the proximal end 2, including at least a part of the straight tube 4, functions as a grip portion. In this way, there is no need for a separate grip to protrude from the proximal end 2 of the instrument 1 or the tube 4 itself, which is a considerable advantage since such a grip could be in the way during surgery and limit the field of applications for the instrument 1. Of course, the proximal end 2 of the instrument 1 could be connected to a separate handle if so desired, for example if the instrument is to be fixedly secured in a rack of some kind. Also, as will be described below, the instrument may be connected to a suction device provided with a handle.

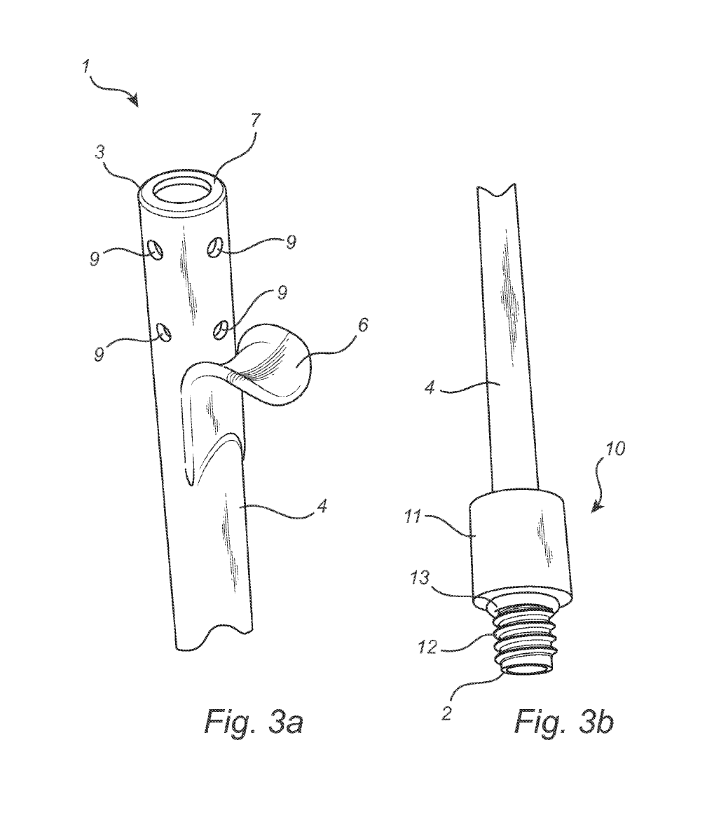

As may be seen more clearly in FIG. 3a, a hook portion 6 is arranged adjacent the distal end 3 of the surgical instrument 1. The hook portion 6 has a saddle shape. The tube 4 extends distally beyond the hook portion 6. In other words, the hook portion 6 is arranged in the proximity of the distal end 3, i.e. at some distance from the outer end 7 of the tube 4.

A number of transversal apertures 9 are formed in the tube 4 adjacent the distal end 3 of the surgical instrument 1. In the embodiment shown, there are two lateral apertures 9 on each side of the tube 4, and two apertures 9 facing upwards and downwards, respectively. The diameter of the apertures 9 may be approximately 1-2 mm, and there may be apertures in an area extending approximately 0.5-1 cm from the outer end 7 of the tube 4.

With reference to FIG. 3b, the surgical instrument 1 is, at the proximal end 2, provided with a coupling portion 10 for connection to a suction device. The suction device may be any suction device suitable for suction during surgery. The coupling portion 10 may be chosen from a set of exchangeable coupling portions, each adapted for a specific type of suction device. When the surgical instrument 1 is connected to the suction device, the tube 4 forms a suction tube. The coupling portion 10 is preferably arranged such that it extends in the direction of the tube 4, as an extension of the tube at the proximal end 2. In this way, one avoids protruding parts or angled sections, deviating considerably from the elongated tube shape, at the proximal end of the instrument. Any such protruding part could be in the way during surgery and limit the field of applications for the instrument 1. The elongated tube shape also facilitates use of one and the same instrument at varying depths into the trocar or incision.

As already mentioned above, the surgical instrument 1 may in addition to, or as an alternative to, suction be used for irrigation. For the sake of clarity of the following description, reference will only be made to suction, but in each instance, irrigation is equally possible. The possibility of using the surgical instrument 1 for irrigation purposes is very important, since irrigation significantly improves the visibility for the operator during surgery.

The coupling portion 10 comprises a rotary coupling 11, such as a swivel, which is threadedly engageable with an outer thread 12 on the proximal end 2 of the surgical instrument 1 in order to allow free rotation of the surgical instrument 1 in relation to a handle of the suction device. The coupling portion 10 further comprises a gasket, such as an o-ring for sealing the connection between the surgical instrument 1 and the suction device.

During laparoscopic surgery, the distal end 3 of the surgical instrument 1 is inserted in the body of a patient through a trocar to a surgical site. The hook portion 6 is used for retracting tissue or an organ from the surgical site where the surgeon performs the actual surgery, using, e.g., scissors, electrosurgical instruments, or grasping instruments. When suction is needed, the hook portion 6 may still be used for retracting the tissue or organ, and the suction device connected to the proximal end 2 of the surgical instrument is activated, such that suction is performed through the outer end 7 of the tube 4. The transversal apertures 9 reduce the risk of getting the instrument stuck, since they allow air to be drawn in even if the outer end 7 of the tube 4 is blocked. When suction has been performed, the suction device is simply deactivated, and the instrument continues to act only as a hook or retractor.

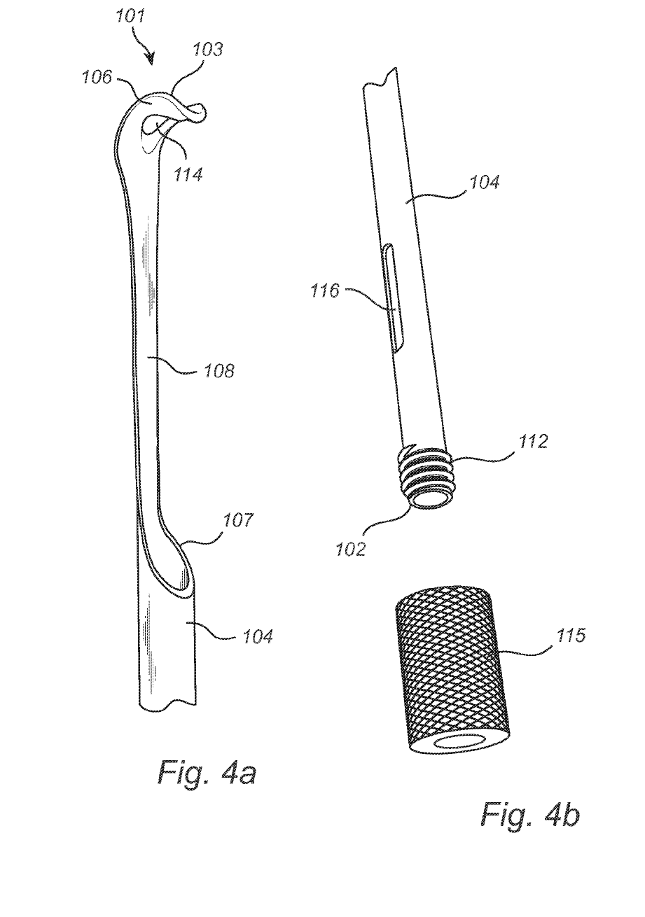

FIG. 2 shows a surgical instrument 101 according to a second embodiment. In the same way as the surgical instrument 1 of the first embodiment, the surgical instrument 101 of the second embodiment has a proximal end 102 and a distal end 103. A major part of the surgical instrument 101 is made up of a tube 104, which extends from the proximal end 102 of the surgical instrument 101 to a location 105 adjacent the distal end 103. As may be seen more clearly in FIG. 4a, a hook portion 106 extends from an outer end 107 of the tube 104 to the distal end 103 of the surgical instrument 101. The hook portion 106 has a narrow stem 108 attached to the outer end 107 of the tube 104. In other words, the hook itself, i.e. the curved part of the hook portion 106, is arranged at some distance from the outer end 107 of the tube 104. In the embodiment shown, the outer end 107 of the tube 104 is oblique in relation to a longitudinal direction of the tube 104. In the hook portion 106, an opening 114 is formed. The opening is dimensioned to allow passage of a suction end of a suction tube inserted in the tube 104 of the surgical instrument 101. The opening 114 may be slightly conical to guide the suction tube into the opening 114.

In the same way as the surgical instrument 1 of the first embodiment, the surgical instrument 101 of the second embodiment may in addition to, or as an alternative to, suction be used for irrigation. Thus, the suction tube inserted in the tube 104 may be used for irrigation as well as for suction. In the same way as above, reference will in the following only be made to suction, although irrigation is equally possible.

At the proximal end 102 of the surgical instrument, a coupling portion 110 is provided. The coupling portion 110 comprises an outer thread 112 onto which a sealing and fixing device 115 is threadable, as may be seen more clearly in FIG. 4b. The coupling portion 110 is preferably arranged such that it extends in the direction of the tube 104, as an extension of the tube at the proximal end 102. In this way, one avoids protruding parts or angled sections, deviating considerably from the elongated tube shape, at the proximal end of the instrument. Any such protruding part could be in the way during surgery and limit the field of applications for the instrument 1. The tube 104 is adapted for insertion of a suction tube of a suction device, and the sealing and fixing device 115 is adapted to seal around the suction tube, and to fix a longitudinal position of the suction tube inside the tube 104 of the surgical instrument 101. The sealing and fixing device 115 may include a slotted rubber body, through which the suction tube is insertable through the slot. The elastic properties of the rubber body may ensure sealing around the suction tube, and if the suction tube is removed from the tube 104 of the surgical instrument 101, the elasticity of the rubber allows the slot to close completely, thereby sealing off the surgical instrument to prevent loss of pneumoperitoneum. The sealing rubber body may also help avoiding involuntary sliding of the suction tube once it is placed in the desired position or retracted to an inactive position inside the tube 104. Thus, repeated insertions and retractions may be avoided. In another embodiment, the elasticity of the rubber may allow the sealing and fixing device 115 to close completely while the suction tube still is inside it, thereby sealing off the surgical instrument without having to remove the suction tube from the tube 104. An orientation indicator 116 in the form of a chamfered portion of the tube 104 is provided near the proximal end 102 of the surgical instrument 101 in order to easily provide the operator with information on the orientation of the surgical instrument 101. The operator may feel the indicator with a tip of a finger to ascertain the orientation of the hook portion 106.

During laparoscopic surgery, the distal end 103 of the surgical instrument 101 is inserted in the body of a patient through a trocar. The hook portion 106 is used for retracting tissue or an organ from the site where the surgeon performs the actual surgery, using, e.g., scissors, electrosurgical instruments or grasping instruments. When suction is needed, the hook portion 106 may still be used for retracting the tissue or organ, and the suction tube of the suction device is inserted in the tube 104, with a suction end of the suction tube extending slightly distally through the opening 114 in the hook portion 106. The operator may choose the distance the suction tube extends distally of the distal end 103 of the surgical instrument 101. The sealing and fixing device 115 may provide a slight resistance to longitudinal movement of the suction tube, such that involuntary movement is prevented. Preferably, the suction tube is inserted in the tube 104 at the start of the surgery, and not withdrawn until the surgical instrument is withdrawn through the trocar. When suction is not needed, the suction tube may be retreated a short distance into the tube 104, and kept in position there by the sealing and fixing device 115, until suction is once more needed, and the suction tube is again advanced, such that the suction end of the suction tube extends out through the opening 114 in the hook portion 106. Suction may be performed at the distal end 103 of the surgical instrument 101, or slightly distally or proximally of the distal end 103.

As used herein the term saddle shape implies a three-dimensional shape resembling a riding saddle. The saddle shape may be said to resemble a hyperbolic paraboloid.

The skilled person realises that a number of modifications of the embodiments described herein are possible without departing from the scope of the invention, which is defined in the appended claims.

For instance, although the surgical instrument of the invention has been described in connection with laparoscopy, it is equally useful for other types of keyhole surgery, such as thoracoscopic surgery. The surgical instrument of the invention is particularly useful as an assistant's instrument in conjunction with robotic surgery. In the context of robotic surgery, the surgical instrument of the invention may advantageously be used by a human assistant, but it may also be operated by a robot. The instrument may also be used for open surgery.

For keyhole surgery, the length of the surgical instrument may, for instance, be approximately 43 cm, and the overall outer diameter may be adapted to trocars of, e.g., 10 mm, 12 mm or 15 mm. The surgical instrument may also be shorter or longer, depending on the intended use. For open surgery, the length of the surgical instrument may be shorter, and the overall outer diameter may be adapted to the particular site of surgery, without being restricted to any trocar diameter. For either kind of surgery, the length of the tube 104 of the second embodiment may be adapted to the length of the suction device of the particular suction device it is used with.

In the first embodiment, the length of the tube 4 that extends distally beyond the hook portion 6 may be longer or shorter than shown in FIG. 3a. For instance, the hook portion may start at a position on the tube which is more proximal than the one shown in FIG. 3a, with a longer length of the tube extending beyond the hook portion.

In the second embodiment, the length of the stem 108 may be chosen depending on the particular use for which the surgical instrument 101 is intended.

The surgical instrument of the invention may be made from medical grade steel, or any other medical grade metal. The metal may advantageously be coated by an electrically non-conductive material, in order to make the instrument safer to use in conjunction with electric cauterisation. It is also possible to make the whole instrument from a medical grade, electrically non-conductive material, such as a suitable plastic material.

The material chosen should be made from a material that can withstand repeated sterilisations, in order to allow multiple use of the instrument. The material should be scratch-resistant and wear resistant. Alternatively, the surgical instrument may be made for single use. In such case, the material need only withstand a single sterilisation.

The instrument may be made with smooth surfaces in order to simplify cleaning. Preferably, the inside of the tube is smooth, such that it is easy to rinse through the tube.

The surgical instrument of the invention may be made with a non-reflective surface, such that glare is avoided. This is particularly useful in keyhole surgery, such as laparoscopy.

The tube may be provided with a measuring scale in an area close to the distal end of the instrument. The measuring scale may be, for instance, 10 cm long, divided into 0.5 and 1 cm intervals. Such a measuring scale is particularly useful during keyhole surgery, where the varying enlargement of the imaging of the surgical site may distort the surgeon's sense of distance. The measuring scale may be printed onto or engraved in the surface of the surgical instrument.

Although an orientation indicator 116 has only been described in connection with the second embodiment, it is equally useful with the first embodiment. Instead of a chamfered portion, the orientation indicator may be constituted by any other tactile means, such as a ridge.

Even though the outer end 107 of the tube 104 in the second embodiment is shown as oblique, it may also be perpendicular to the longitudinal direction of the tube 104.

Instead of a saddle shape as described above, the hook portion of either embodiment may instead have a T shape or a U shape.

In the first embodiment, the transversal apertures 9 may be varied in number and configuration. For instance, transversal apertures may be formed in the entire area from the distal end 3 to a position on the tube 4 where the hook portion 6 starts, or from the distal end 3 to a position on the tube 4 where the hook portion 6 ends.

1. A surgical instrument having a proximal end and a distal end, comprising a hook portion, arranged at said distal end for retracting tissue and/or organs during surgery, a grip portion, arranged at said proximal end for holding said instrument during surgery, and an elongate tube adapted for suction and/or irrigation,
wherein said tube being straight and extending essentially along a length of said surgical instrument from said proximal end in a direction towards said distal end, and
said grip corresponding to a portion of said straight tube.

2. The surgical instrument as claimed in claim 1, wherein said tube extends from said proximal end to said distal end.

3. The surgical instrument as claimed in claim 2, further comprising a coupling portion arranged at said proximal end, for connection to a suction device and/or irrigation device, said tube forming a suction and/or irrigation tube.

4. The surgical instrument as claimed in claim 3, wherein said coupling portion comprises a rotary coupling for allowing rotation of said surgical instrument in relation to said suction device and/or irrigation device.

5. The surgical instrument as claimed in claim 2, wherein said hook portion is arranged adjacent said distal end, and wherein said tube extends distally beyond said hook portion.

6. The surgical instrument as claimed in claim 1, wherein said tube extends from said proximal end to a location proximal of, and adjacent to, said distal end.

7. The surgical instrument as claimed in claim 6, wherein said hook portion extends from an outer end of said tube to said distal end of said surgical instrument.

8. The surgical instrument as claimed in claim 6, wherein said tube is adapted for insertion of a suction tube and/or irrigation tube.

9. The surgical instrument as claimed in claim 8, wherein said hook portion comprises an opening allowing passage of a suction and/or irrigation end of said suction tube and/or irrigation tube.

10. The surgical instrument as claimed in claim 8, further comprising a retaining device arranged to fix a longitudinal position of said suction and/or irrigation tube inside said tube.

11. The surgical instrument as claimed in claim 8, further comprising a sealing device for sealing said tube around said suction and/or irrigation tube.

12. The surgical instrument as claimed in claim 11, said sealing device being arranged to seal said tube when said suction and/or irrigation tube is removed from said tube.

13. The surgical instrument as claimed in claim 11, wherein said sealing device is further arranged to fix a longitudinal position of said suction and/or irrigation tube inside said tube.

14. The surgical instrument as claimed in claim 1, wherein said hook portion has a saddle shape.
